(19) United States
(12) Patent Application Publication
(10) Pub. No.: US 2021/0277360 A1
(43) Pub. Date: Sep. 9, 2021

(54) CELL LINKERS FOR HETEROTYPIC CELL SPHEROIDS

(71) Applicants: Kansas State University Research Foundation, Manhattan, KS (US); The United States of America, as represented by the Secretary, Department of Health and Human Servic, Bethesda, MD (US)

(72) Inventors: Thu Annelise Nguyen, Amarillo, TX (US); Ralph E. Parchment, Frederick, MD (US); Robert J. Kinders, Walkersville, MD (US)

(21) Appl. No.: 17/260,365

(22) PCT Filed: Jul. 15, 2019
(86) PCT No.: PCT/US2019/041800

§ 371 Date: Jan. 14, 2021

Related U.S. Application Data
(60) Provisional application No. 62/698,602, filed on Jul. 16, 2018.

(51) Int. Cl. C12N 5/071; C12N 5/00; C12N 15/10; G01N 33/50; C07K 5/09; C07K 7/06; C12N 5/09

(52) U.S. Cl. CPC C12N 5/0697; C12N 5/0062; C12N 15/1093; G01N 33/5011; G01N 33/5088; C12N 2513/00; C07K 7/06; C12N 5/0693; C12N 2533/50; C12N 2502/30; C12N 2502/28; C07K 5/0817

(57) ABSTRACT

A three-dimensional heterogenous cell spheroid comprising a plurality of at least two different types of cells and one or more short peptide cell linkers. The peptide cell linkers are linear peptides having respective terminal ends, and comprising an adhesion sequence at or near each end, with a suitable spacer sequence between each adhesion sequence. Methods of making and using such spheroids for in vitro testing are also described.

### CROSS-REFERENCE TO RELATED APPLICATIONS

The present application claims the priority benefit of U.S. Provisional Patent Application Ser. No. 62/698,602, filed Jul. 16, 2018, entitled CELL LINKERS FOR HETEROTYPIC CELL SPHEROIDS, incorporated by reference in its entirety herein.

### FEDERALLY SPONSORED RESEARCH OR DEVELOPMENT

This invention was made with government support under National Cancer Institute Contract No. HHSN-261200800001E awarded by National Institutes of Health. The government has certain rights in the invention.

### SEQUENCE LISTING

The following application contains a sequence listing in computer readable format (CRF), submitted as a text file in ASCII format entitled “Sequence Listing,” created on Jul. 15, 2019, as 4 KB. The content of the CRF is hereby incorporated by reference.

### BACKGROUND OF THE INVENTION

### Field of the Invention

The present invention relates to heterotypic spheroids comprising peptide-based cell crosslinkers for in vitro test systems.

### Description of Related Art

Three-dimensional (3D) cell culture models have become increasingly popular and are thought to be a more accurate physiological representation of the in vivo situation as compared to cells grown in two-dimensional monolayers, where many cellular characteristics are impaired due to artificial conditions. 3D model systems can better reflect the histological, biological and molecular characteristics of, e.g., primary tumors with its tissue-specific architecture. Whereas two-dimensional assays cannot reproduce the complexity and heterogeneity of various conditions, such as cancer, in vivo and often fail to predict the effects of compounds added to the system for later in vivo application. However, many 3D models are still limited and fail to account for cell-to-cell interactions between different cell types in vivo. At the present time, no material or process exists to create 3D mixed spheroids of different cell types (e.g., endothelial and epithelial cells) within the same spheroid, since these two cell types do not form close associations in vitro.

### SUMMARY OF THE INVENTION

The present invention is broadly concerned with improved 3D spheroids comprising at least two different cell types to better reflect in vivo cell systems for study and in vitro testing. In one aspect, described herein are heterogenous cell spheroids comprising a plurality of at least two different types of cells (e.g., different cell lines, histological or phenotypic origins, etc.) and one or more peptide cell linkers. Advantageously, in the approaches used herein the cells seeded onto culture plates proliferate into the spheroids. Each of the peptide cell linkers is a linear peptide having respective terminal ends, and comprising an adhesion sequence at or near each end (i.e., within 3-5 residues). Preferably, each of the peptide cell linkers further comprises a spacer sequence between each adhesion sequence.

In one or more aspects, the different cell types are selected from the group consisting of mesenchymal, epithelial cell, endothelial cells, stromal cells, tumor cells, stem cells, fibroblasts, macrophages, dendritic cells, adipocytes, and combinations thereof. As demonstrated by the data, the different cell types are substantially uniformly distributed throughout the body of the spheroid. In one or more embodiments, spheroids can be prepared comprising at least one type of cancer cell and one type of non-cancer cell. Spheroids can be prepared according to various desired characteristics, but to prevent core cell death and have optimum function, the spheroids will preferably have a diameter (e.g., maximum surface to surface dimension) of less than 500 microns, preferably less than 300 microns, more preferably from about 50 to about 200 microns, even more preferably from about 100 to about 200 microns.

As described herein are methods of forming spheroids according to various embodiments of the invention. The methods generally comprise preparing a cell suspension comprising a plurality of at least two different types of cells in a suitable cell culture medium, which is then mixed with one or more peptide cell linkers. The resulting cell mixture is then preferably cultured in a hydrogel scaffold for spheroid formation. In preferred embodiments, the cell mixture is dispersed with a peptide suspension comprising a plurality of hydrogel-forming peptides (preferably amphiphilic and self-assembling linear peptides). The peptide suspension is then allowed to form a hydrogel, which can include addition of crosslinkers, or reliance on components of the cell media suspension (e.g., ions, serum, etc.) to initiate gelation. The cell mixture is cultured in the hydrogel under favorable conditions for cell proliferation and expansion into a population of spheroids, where each of the spheroids comprises a plurality of at least two different types of cells and one or more peptide cell linkers. The methods can further comprise collecting the spheroids from the hydrogel. In one or more embodiments, the hydrogel is a shear thinning gel. In one or more embodiments, the spheroids can be collected by applying a kinetic force to the hydrogel (e.g., centrifugation, pipetting, etc.), which essentially reverses the hydrogel (i.e., liquefies it) to easily release the spheroids without damage to the spheroid body(ies).

Methods of using the spheroids are also described herein. It will be appreciated that virtually any in vitro testing can be carried out with the spheroids. In one or more embodiments, the methods comprise providing a population of spheroids as described herein, and contacting the spheroids with a compound of interest (e.g., active agent, bioactive molecules, therapeutic or diagnostic reagents, biologics, xenobiotics, new chemical entities, etc.) for a period of time, and detecting the effect of the compound of interest on the spheroid or the cells therein. In some cases, a differential effect of the compound of the different cell types in the spheroid may be detected. The approaches of the invention are particularly suited for cancer drug screening. In one or more embodiments, the spheroid comprises at least one type of tumor cell, and the compound of interest can be a chemotherapeutic. Thus, the spheroids can be used to determine if the compound of interest can kill the cancer cells. Personalized spheroids based upon patient tissue samples can be made using the inventive process to provide individualized compound screening targeted to the patient's exact tumor cell type(s), such as by collecting a tissue sample from an identified patient's tumor or cancer-containing biological samples (e.g., blood, lymph, etc.), isolating cancer cells from the sample, and using these cells to create spheroids for screening against a variety of potentially therapeutic compounds.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1. Peptide hydrogelation in MEM. A. Proposed mechanism of MEM-induced PGMatrix™ hydrogel (h9e) peptide self-assembling hydrogelation (SEM image showing the nanofiber scaffold of the hydrogel matrix). B. Storage modulus G′ of 1, 2, and 3 mM peptide hydrogel during the hydrogelation at 37° C. C. SEM image of 1 mM peptide hydrogel. D. SEM image of 3 mM peptide hydrogel.

FIG. 2. Multiple times delivery of peptide hydrogel via pipette. Hydrogel was shear thinning but reassembled quickly without permanently destroying hydrogel architecture.

FIG. 3. Immunofluorescence image of Epithelial-Endothelial 3D spheroids of heterotypic cells DU145 and HUVEC at Day 7 treated with 0.1 μM Nilotinib for 6 days.

FIG. 4. Immunofluorescence image of Epithelial-Endothelial 3D spheroids of heterotypic cells DU145 and HUVEC at Day 7 without any treatment.

FIG. 5. Immunofluorescence image of Epithelial-Endothelial 3D spheroids of heterotypic cells MDA-MB-468 and HUVEC at Day 7, treated with 0.1 μM Nilotinib for 6 days.

FIG. 6. Immunofluorescence image of 5 μm thickness sections of Epithelial-Endothelial 3D spheroids of heterotypic cells DU145 and HUVEC treated with 0.5 μM Erlotinib and/or 0.5 μM Cediranib for 6 days.

FIG. 7A. Imaging of DU145 cells cultured under control conditions (without linker), with and without HUVEC.

FIG. 7B. Imaging of DU145 cells cultured with linker #1, with and without HUVEC.

FIG. 7C. Imaging of DU145 cells cultured with linker #2, with and without HUVEC.

FIG. 7D. Imaging of DU145 cells cultured with linker #3, with and without HUVEC.

FIG. 7E. Imaging of DU145 cells cultured with linker #4, with and without HUVEC.

FIG. 7F. Imaging of DU145 cells cultured with linker #5, with and without HUVEC.

FIG. 7G. Imaging of DU145 cells cultured with linker #6, with and without HUVEC.

FIG. 7H. Imaging of DU145 cells cultured with linker #7, with and without HUVEC.

FIG. 7I. Imaging of DU145 cells cultured with linker #8, with and without HUVEC.

FIG. 8A. Imaging of MDA-MB-468 cells cultured under control conditions (without linker), with and without HUVEC.

FIG. 8B. Imaging of MDA-MB-468 cells cultured with linker #1, with and without HUVEC.

FIG. 8C. Imaging of MDA-MB-468 cells cultured with linker #2, with and without HUVEC.

FIG. 8D. Imaging of MDA-MB-468 cells cultured with linker #3, with and without HUVEC.

FIG. 8E. Imaging of MDA-MB-468 cells cultured with linker #4, with and without HUVEC.

FIG. 8F. Imaging of MDA-MB-468 cells cultured with linker #5, with and without HUVEC.

FIG. 8G. Imaging of MDA-MB-468 cells cultured with linker #6, with and without HUVEC.

FIG. 8H. Imaging of MDA-MB-468 cells cultured with linker #7, with and without HUVEC.

FIG. 8I. Imaging of MDA-MB-468 cells cultured with linker #8, with and without HUVEC.

FIG. 9A. Imaging of COLO 205 cells cultured under control conditions (without linker), with and without HUVEC.

FIG. 9B. Imaging of COLO 205 cells cultured with linker #1, with and without HUVEC.

FIG. 9C. Imaging of COLO 205 cells cultured with linker #2, with and without HUVEC.

FIG. 9D. Imaging of COLO 205 cells cultured with linker #3, with and without HUVEC.

FIG. 9E. Imaging of COLO 205 cells cultured with linker #4, with and without HUVEC.

FIG. 9F. Imaging of COLO 205 cells cultured with linker #5, with and without HUVEC.

FIG. 9G. Imaging of COLO 205 cells cultured with linker #6, with and without HUVEC.

FIG. 9H. Imaging of COLO 205 cells cultured with linker #7, with and without HUVEC.

FIG. 9I. Imaging of COLO 205 cells cultured with linker #8, with and without HUVEC.

FIG. 10A. Imaging of PANC-1 cells cultured under control conditions (without linker), with and without HUVEC.

FIG. 10B. Imaging of PANC-1 cells cultured with linker #1, with and without HUVEC.

FIG. 10C. Imaging of PANC-1 cells cultured with linker #2, with and without HUVEC.

FIG. 10D. Imaging of PANC-1 cells cultured with linker #3, with and without HUVEC.

FIG. 10E. Imaging of PANC-1 cells cultured with linker #4, with and without HUVEC.

FIG. 10F. Imaging of PANC-1 cells cultured with linker #5, with and without HUVEC.

FIG. 10G. Imaging of PANC-1 cells cultured with linker #6, with and without HUVEC.

FIG. 10H. Imaging of PANC-1 cells cultured with linker #7, with and without HUVEC.

FIG. 10I. Imaging of PANC-1 cells cultured with linker #8, with and without HUVEC.

FIG. 11A. Imaging of HT29 cells cultured under control conditions (without linker), with and without HUVEC.

FIG. 11B. Imaging of HT29 cells cultured with linker #1, with and without HUVEC.

FIG. 12A. Imaging of CAKI-1 cells cultured under control conditions (without linker), with and without HUVEC.

FIG. 12B. Imaging of CAKI-1 cells cultured with linker #1, with and without HUVEC.

FIG. 12C. Imaging of CAKI-1 cells cultured with linker #2, with and without HUVEC.

FIG. 13A. Western blot images of DU145 cell spheroids.

FIG. 13B. Western blot images of MDA-MB-468 cell spheroids.

FIG. 13C. Western blot images of COLO 205 cell spheroids.

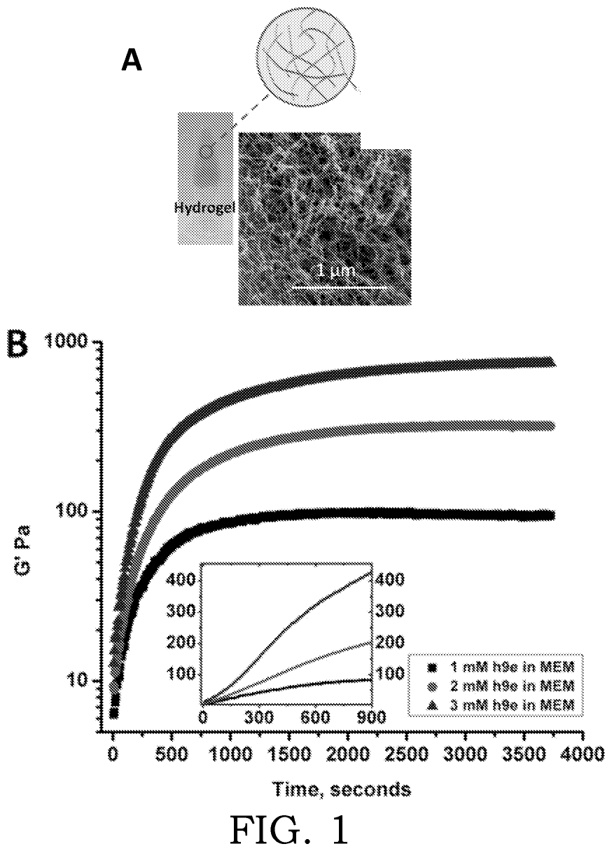

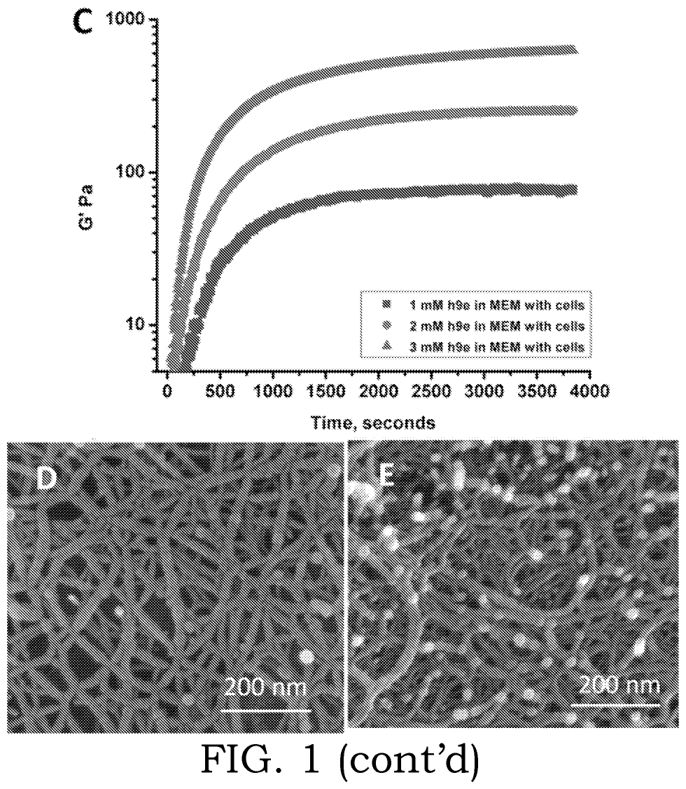

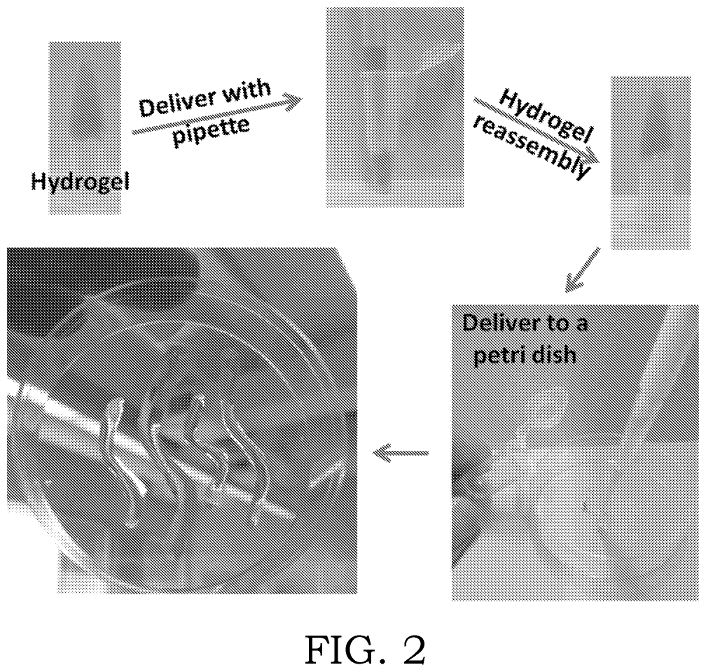

### DETAILED DESCRIPTION

The present invention is concerned with 3-dimensional multicellular spheroids comprising at least two different types of cells and one or more peptide cell linkers. As such, the resulting spheroids are heterotypic (aka heterogenous) and can be used to study heterocellular interactions between two or more cell types in a 3-dimensional in vitro system. “Spheroids” are clusters of cell colonies, formed by self-assembly when cell-to-cell interactions overcome cell-substrate interactions. Spheroids may be generally spherical, but may also have an irregular 3-dimensional shape.

The peptide cell linkers are small synthetic peptide sequences comprising at least two different cell adhesion peptide sequences (peptide ligands) so as to promote interaction and association of the two or more different cell types for cellular growth and expansion into heterogenous spheroids. In one or more embodiments, the peptide cell linker will be a linear (unbranched) peptide comprising a cell adhesion peptide sequence at or near each terminal end (C-terminal and N-terminal), with a spacer sequence in between the adhesion sequences. The peptide cell linkers are generally short linear peptides of at least 9 amino acid residues and no more than 50 amino acid residues or less in length, preferably 40 residues or less, more preferably 30 residues or less, more preferably 25 residues or less, even more preferably 23 residues or less, even more preferably from 9 to 23 residues. Thus, it is envisioned that peptide sequences of only 9 residues could be used (e.g., 3-amino acid peptide ligands at each end with an intervening 3-amino acid spacer sequence). It will be appreciated that the use of short, defined, synthesizable peptides with linkers is a significant advantage with relatively low cost, but high flexibility for modification to optimize the peptides for particular cell binding. In general, the cell adhesion peptide sequences will be from about 2 about 8 amino acids in length, preferably from about 3 to about 5 amino acids in length. The spacer sequence will generally be of adequate length to maintain separation of the respective cell adhesion sequences without folding in on itself, but be short enough to bring the target cells into proximity for spheroid formation and cell-to-cell interaction. In general, the spacer sequence will be from about 3 about 10 amino acids in length, preferably from about 5 to about 8, and even more preferably from about 6-7 amino acids in length. The amino acids used in the spacer sequence are preferably designed based upon the cell culture medium, and specifically to ensure that the peptide linker will be water soluble and able to blend into the cell culture media. The peptide cell linkers preferably have a MW in the range of from about 500 to about 3000 Daltons, more preferably from about 600 to about 2500, more preferably from about 750 to about 2500 Daltons.

By way of example, the specific design of a 20-amino acid peptide linker for endothelial and epithelial cells, bringing two cell types in proximity of each other for cell growth expansion. The peptide sequence is designed and synthesized with specific recognition/adhesion sequences for endothelial cell surfaces at one end and epithelial cell surfaces at the other end. Thus, the recognition/adhesion sequences are peptide ligands with high affinity binding for their respective designated cell type. As used herein, “high affinity binding” means the ligand has a strong and specific affinity for its target cell type (and specifically cell surface receptors), generally characterized by a binding reaction with a minimum equilibrium dissociation constant (K_{d}) of 10e-6. However, it will be appreciated that smaller peptide ligands, if selected for the adhesion sequence, will inherently bind with lower affinity. This may be an advantage under certain reaction conditions, so long as concentrations of the ligand and receptor are high enough in the cell culture. Further, it will be appreciated that the relative position and length of the sequences in the peptide are important aspects of the design.

In one or more embodiments, the peptide cell linker is a synthetic peptide sequence of FLIYIGSRIGPGGDGPGGRGD (SEQ ID NO:5), which recognizes endothelial and epithelial cell surfaces. This particular peptide linker was designed based upon a peptide hydrogel cell culture media PGMatrix™. In one or more embodiments, the peptide hydrogel cell culture media comprises a peptide of the sequence FLIVIGSIIGPGGDGPGGD (“h9e”; SEQ ID NO:14). Utilizing this sequence as the basic building block, new peptide sequences were synthesized to target endothelial cells (using adhesion sequence YIGSR, SEQ ID NO:1) and epithelial cells (using adhesion sequence RGD). Thus, in one or more embodiments, the peptide cell linker is FLIYIGSRIGPGGDGPGGRGD (SEQ ID NO:5), where the bold residues are the adhesion sequences and IGPGGDGPGG (SEQ ID NO:2) is the spacer sequence. Important residues of the sequence are YIGSRIGPGPGGRGD (SEQ ID NO:9), in which the spacer is shortened to a 7 amino acid length between the two targets as well as water solubility to blend in with the PGMatrix™. It will be appreciated that other peptide cell linkers can be designed and synthesized applying the above-described principles, as exemplified in the working examples.

Exemplary adhesion sequences comprise (consist, or consist essentially of) YIGSR (SEQ ID NO:1), RGD, KGD, and/or LDV. In one or more embodiments, peptide cell linkers are designed for targeting endothelial cells using adhesion sequence YIGSR (SEQ ID NO:1), for stromal cells using adhesion sequences KGD or LDV, or for targeting epithelial cells using adhesion sequence RGD. For example, peptide cell linker FLIYIGSRIGPGGDGPGGKGD (SEQ ID NO:6) is exemplary for targeting endothelial cells and stromal cells. Peptide cell linker LDVIGPGGDGPGGRGD (SEQ ID NO:8) is also exemplary for targeting endothelial and stromal cells, while LDVGPGGDGPGGRGD (SEQ ID NO:11) is exemplary for targeting stromal and epithelial cells. Exemplary peptide cell linkers comprise (consist, or consist essentially of) the following sequences:

(SEQ ID NO: 5)
FLIYIGSRIGPGGDGPGGRGD
(SEQ ID NO: 6)
FLIYIGSRIGPGGDGPGGKGD
(SEQ ID NO: 7)
FLIYIGSRIGPGGDGPGGLDV
(SEQ ID NO: 8)
LDVIGPGGDGPGGRGD
(SEQ ID NO: 9)
YIGSRIGPGPGGRGD
(SEQ ID NO: 10)
RGDIGPGGDGPGGRGD
(SEQ ID NO: 11)
LDVGPGGDGPGGRGD
or
(SEQ ID NO: 12)
FLIYIGSRIGPGGDGPGGYIGSR,

where the bold residues are the adhesion sequences and IGPGGDGPGG (SEQ ID NO:2), IGPGPGG (SEQ ID NO:3), and GPGGDGPGG (SEQ ID NO:4) are spacer sequences. It will be appreciated that the particular adhesion sequences can be modified and optimized for additional vertebrate species. Further, the terminal position of the adhesion sequences can be reversed from the sequences shown above. That is, the designated adhesion sequence could be positioned at either the N-terminus or C-terminus in the foregoing sequences, depending upon the particular linker. For example, LDVIGPGGDGPGGRGD (SEQ ID NO:8) could instead be RGDIGPGGDGPGGLDV (SEQ ID NO:13) without departing from the scope of the invention. Further, as demonstrated by certain sequences above, one to three additional residues may be added to the adhesion sequence at either terminal end. It is also foreseeable that a third or nth adhesion sequence could be inserted in the middle of the spacer sequence if desired.

Regardless of the embodiment, the two or more different cell types are co-cultured with the peptide cell linkers under suitable conditions for proliferation of the cells into the desired spheroids. Thus, unlike prior approaches for making 3-dimensional spheroids that involve mere aggregation of the seeded cells into spheroids or organoids, the current invention involves proliferation and expansion of the cultured cells into spheroids. In other words, total cell numbers in the resulting spheroid population is increased as compared to the number of cells originally seeded.

Preferably, at least one cell type in the heterogenous spheroids are proliferating cells. Thus, cells in the inventive heterogenous spheroids have active proliferation and metabolism, and exhibit heterotypic cell-to-cell interactions. This is a particularly important feature of the inventive spheroids. This is because abnormal cell behavior in vivo, such as growth and metastasis of tumor cells is often caused by inappropriate cell-to-cell interactions within the microenvironment. However, current 3-dimensional approaches for in vitro cancer testing often rely on homogenous spheroids formed from the target tumor cells only. Accordingly, the results obtained by such testing do not accurately reflect the true cell interactions occurring within the patient. Further, even outside of cancer research, homogenous spheroids have known limitations for examining pharmacodynamics related to the interface of heterogenous cell populations. Thus, the inventive heterogenous spheroids provide a number of conveniences and advantages over homogenous spheroids.

A variety of cells can be used in the invention for creating the heterogenous spheroids. Exemplary cell sources include tissues such as fresh tissue, cryopreserved tissue, cell culture lines, genetically engineered cells from any source, and the like. The starting material can be derived from pure or semi-pure fractions of individual cells types. The cells may be derived (directly or indirectly) from any suitable human or animal subject or patient, including human, canine, feline, bovine, equine, porcine, simian, and murine sources, among others. Primary cells may be used as well as established cell lines. Primary cells are those taken directly from benign or malignant tissue from a subject (e.g., biopsy material) and established for growth in culture (in vitro). Cell lines refers to cells that have been genetically modified and/or have been continually passaged several times so as to have acquired homogenous genotypic and phenotypic characteristics. Cell lines can be finite or continuous (immortalized cells), meaning they may continue to grow permanently in cell culture under suitable culture conditions. Cell types for use in the spheroids include epithelial cells, stromal cells, endothelial cells, mesenchymal cells, stem cells, tumor cells, fibroblasts (including cancer associated fibroblasts), adipocytes, dendritic cells, macrophages, and the like. Tumor cells that can be included in the spheroids include patient's primary tumor cells, immortalized or transformed cells, tumor cell lines. Exemplary cell lines for use in the spheroids include, without limitation, MDA-MB-231, HT29, Colo205, DU145, MDA-MB-468, CAKI-1, PANC-1, HUVEC, and the like.

In one or more embodiments, the cells are co-cultured along with the peptide linkers in a suitable culture media and cell culture environment allowing for the formation of a spheroid. A variety of techniques are known for creating spheroids, including hanging drop technique, microwells, and scaffolds, and the like. In one or more embodiments, the culture media comprises a hydrogel matrix forming material, which provides support and a scaffold to allow 3-dimensional spheroid formation. The hydrogel structure, morphology, and components can be tuned to the particular needs of the cells. As noted, a preferred hydrogel-based cell culture media is PGMatrix™, which comprises self-assembling amphiphilic peptides that form shear thinning hydrogel matrices. Such peptides and their associated hydrogels are described in detail in U.S. Pat. No. 8,835,395, “Novel Protein Peptide Hydrogels,” filed Mar. 10, 2011, and incorporated by reference herein in its entirety. Additional components necessary for the survival cell proliferation (inorganic salts, carbohydrates, hormones, essential amino acids, non-essential amino acids, vitamins), basal medium (e.g., Dulbecco's Modified Eagle medium (DMEM), Minimum Essential medium (MEM), RPMI), and the like can also be included.

The cell media hydrogel can be formed by providing a solution of the peptides suspended, dispersed, or dissolved in a solvent (preferably water). Gelation can be triggered by adding source of ions into the peptide solution, with preferred ions being selected from the group consisting of ions of Group I and Group II metals. This is referred to herein as the “ion trigger method.” The most preferred Group I and Group II metal ions are selected from the group consisting of Ca, Na, Mg, K, and Zn ions. Exemplary sources of these ions include Group I and Group II metal chlorides, Group I and Group II metal bromides, Group I and Group II metal sulfides, Group I and Group II metal carbonates and bicarbonates. Albumin, such as found in serum, can also be used to trigger gelation. A combination of protein and/or ion can also be used, depending upon the particular morphology and strength of the hydrogel desired. In any case, the gel is considered formed once G′ (storage modulus) is greater than G″ (storage loss).

The peptide-based hydrogels are characterized by a uniform internetwork morphology with a porous structure and open cells. The gel will comprise peptide nanofibers having an average diameter of from about 3 nm to about 30 nm, preferably from about 5 nm to about 20 nm, and more preferably from about 8 nm to about 15 nm, as measured under a transmission electron microscope. The gel will include peptide nanofibers having an average length of from about 0.3 μm to about 5 μm, preferably from about 0.8 μm to about 3 μm, and more preferably from about 1 μm to about 2 μm. Advantageously, the peptide hydrogel displays shear-thinning and repeatedly reversible sol-gel transfer properties that enable it to be easily transferred via an injector. As used herein, references to the hydrogel being “shear thinning” means that the viscosity of the gel decreases with an increase in the rate of shear stress. This is a particularly advantageous property for subsequent isolating the cultured spheroids from the matrix. In one or more embodiments, centrifugation or pipetting can be used to disturb and liquefy the matrix to easily release the spheroids. On certain days during the spheroid formation process hormones or other proteins or scaffold-enhancing products could also be added to the media to enhance association of the cells and spheroid development.

It will be appreciated that the parameters for the cell culture can be varied. Different ratios of the cells can be used, along with different corresponding ratios of peptide linkers. The components of the peptide hydrogel itself can also be adjusted for a stronger or weaker gel, as well as to modulating the speed of hydrogel formation (e.g., slow vs. fast). Exemplary ratios include a ratio of epithelial cells to endothelial cells of 1:3 to 3:1, 1:2 to 2:1, and even 1:1, in cell culture media (supplemented with serum), with 1% (vol) peptide linker in aqueous solution. This mixture is added to the peptide hydrogel solution, and the resulting combination is plated to allow hydrogel formation with the cells embedded in the matrix. The cells proliferate in the matrix, and the peptide linker draws the different types of cells together, which ultimately form the spheroids.

The resulting heterogenous spheroids exhibit characteristics that substantially mimic those of the tissue of origin, including cell-to-cell microenvironments. The value of this approach is the ability to more closely simulate interactions common in living organisms (in vivo) compared to what is currently state of the art in simple systems (in vitro) commonly employed for testing of, among others, drug activity, particularly where interaction of endothelial and epithelial cells affects the anticipated or desired drug activity. An immediate application of this technology with commercial utility is in anti-cancer drug screening, including disease such as colon cancer, in which interaction of colon tumor cells with mesenchymal cells is a proven method of tumor growth regulation.

Since the heterogenous spheroids substantially mimic in vivo cell systems, these spheroids can thus be used for diagnostic and/or therapeutic purposes, for example, drug testing, drug screening, pharmacokinetic/dynamic profiling, toxicity studies, personalized therapies, biomarker identification, high throughput screening/drug identification, and the like. The heterogenous spheroids can also be used for the screening of potentially therapeutic agents for an effect on at least one type of tumor cell. For example, spheroids can be incubated with a candidate compound, and monitored for the effect of the compound on spheroid growth and/or cell viability.

Embodiments of the invention are also concerned with methods for production of heterogenous spheroids as described above. Preparing a cell suspension comprising at least two different cell types along with cell media components and the peptide linkers. The cell suspension is mixed with the hydrogel-forming peptides described above. Hydrogel formation can then be initiated. This can be based upon components present in the cell suspension itself. Additional crosslinking agents can also be used. The cells are allowed to proliferate and grow in the hydrogel under conditions favorable for spheroid formation, followed by separation of the spheroids from the hydrogel matrix.

Additional advantages of the various embodiments of the invention will be apparent to those skilled in the art upon review of the disclosure herein and the working examples below. It will be appreciated that the various embodiments described herein are not necessarily mutually exclusive unless otherwise indicated herein. For example, a feature described or depicted in one embodiment may also be included in other embodiments, but is not necessarily included. Thus, the present invention encompasses a variety of combinations and/or integrations of the specific embodiments described herein.

As used herein, the phrase “and/or,” when used in a list of two or more items, means that any one of the listed items can be employed by itself or any combination of two or more of the listed items can be employed. For example, if a composition is described as containing or excluding components A, B, and/or C, the composition can contain or exclude A alone; B alone; C alone; A and B in combination; A and C in combination; B and C in combination; or A, B, and C in combination.

The present description also uses numerical ranges to quantify certain parameters relating to various embodiments of the invention. It should be understood that when numerical ranges are provided, such ranges are to be construed as providing literal support for claim limitations that only recite the lower value of the range as well as claim limitations that only recite the upper value of the range. For example, a disclosed numerical range of about 10 to about 100 provides literal support for a claim reciting “greater than about 10” (with no upper bounds) and a claim reciting “less than about 100” (with no lower bounds).

### EXAMPLES

The following examples set forth methods in accordance with the invention. It is to be understood, however, that these examples are provided by way of illustration and nothing therein should be taken as a limitation upon the overall scope of the invention.

### Example 1

PGMatrix™ is a hydrogel available from PepGel LLC (Manhattan, Kans.) and comprises 1% W/V peptide nanofibrils that can be initiated into shear thinning gel. PGMatrix™ solution is warmed to room temperature. For the general protocol, isolated cells are suspended in the selected cell culture medium (e.g., DMEM, RMPI, etc.). PGMatrix™ solution is added to the cell suspension at desired mixing ratio, and then transferred to a cell culture plate. The plate is placed in an incubator at 37° C. 5% CO₂ for 30-60 minutes to complete gelation of the matrix. If needed, additional crosslinking solution (PGWorks) can be added. Serum (FBS) can also be used to trigger or enhance hydrogel formation, if not included in the cell medium. After gelation, a thin layer of cell medium can be added to the surface of the gel to prevent drying. The top medium can be changed every other day to provide fresh nutrition and prevent drying of the hydrogel during the 3D cell culture process, if needed.

In the current work, cells were cultured in 1 mM PGMatrix™ (peptide hydrogel matrix, PepGel, LLC) with 10% FBS media. After 7 days of incubation, the formation of ˜150-micron 3D spheroids were generated to be used for biological assays.

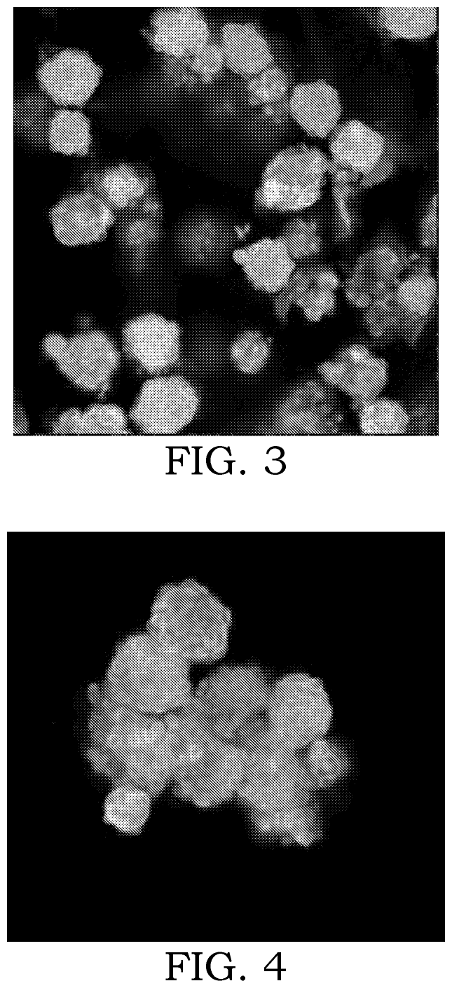

For immunofluorescence assay, 150-micron 3D spheroids were isolated by using centrifugation of the 3D culture. The PGMatrix™ has the unique ability to change from a gelled state to a liquid state by kinetic/motion. Thus, shear thinning/centrifugation can be used to liquefy the media and isolate the spheroids from the matrix. Biomarkers of interest (vimentin, beta catenin, and cadherin) were then stained on the spheroids and images. Immunofluorescence assay was performed using DAPI, staining nuclei (blue), E-cadherin antibodies (green), β-catenin antibodies (orange), and vimentin antibodies, staining endothelial cells (red). The results are shown in FIGS. 3-6.

In one experiment, a total of 100,000 cells of epithelial prostate cancer cells, DU145, and endothelial cells, HUVEC, at 3:1 ratio, respectively, were seeded in 12-well plate with 1 ml total volume media, containing 1 mM PGMatrix™ with mixed media of M200 and RPMI along with 1% peptide linkers. Additional 1 ml of media was added to the top of the gelated cell culture. Culture was treated with various anticancer drugs over a period of 7 days. For the non-treatment control, the top media was changed every two days. FIG. 3 is an image of DU145-HUVEC 3D spheroids captured at Day 7, treated with 0.1 μM Nilotinib for 6 days. FIG. 4 is an image of DU145-HUVEC 3D spheroids captured at Day 7 without any treatment.

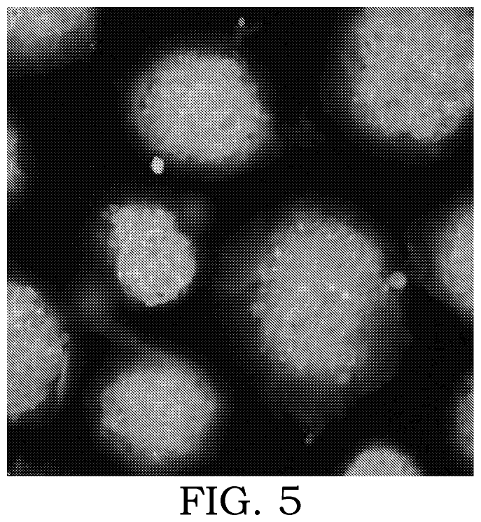

A similar protocol was carried out with a total of 100,000 cells of epithelial prostate cancer cells, MDA-MB-468, and endothelial cells, HUVEC, at 3:1 ratio, respectively, seeded in 12-well plate with 1 ml total volume media, containing 1 mM PGMatrix™ with mixed media of M200 and RPMI along with 1% peptide linkers. Additional 1 ml of media was added to the top of the gelated cell culture. Top media was changed every 2 days. Culture was treated with various anticancer drugs over a period of 7 days. Immunofluorescence assay was performed using DAPI, staining nuclei (blue), E-cadherin antibodies (green), β-catenin antibodies (orange), and vimentin antibodies, staining endothelial cells (red). FIG. 5 is an image of MDA-MB-468-HUVEC 3D spheroids captured at Day 7, treated with 0.1 μM Nilotinib for 6 days.

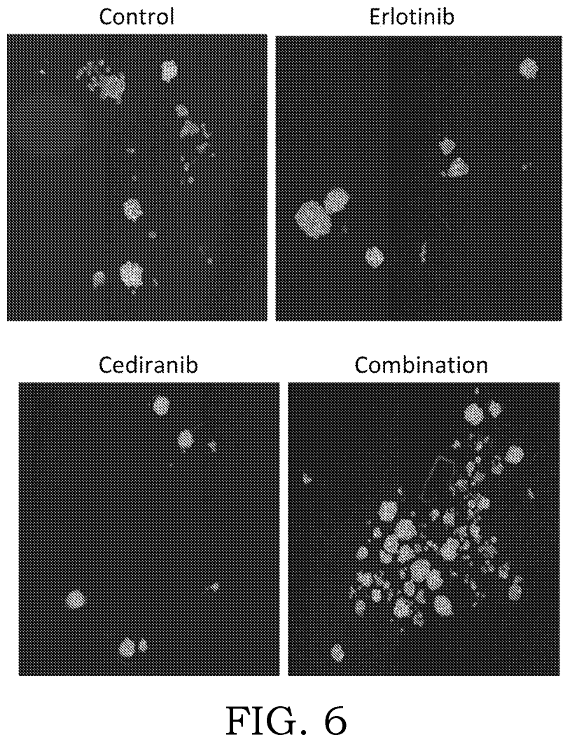

In another experiment, a total of 100,000 cells of epithelial prostate cancer cells, DU145, and endothelial cells, HUVEC, at 3:1 ratio, respectively, was seeded in 12-well plate with 1 ml total volume media, containing 1 mM PGMatrix™ with mixed media of M200 and RPMI along with 1% peptide linkers. Additional 1 ml of media was added to the top of the gelated cell culture. Culture was treated with various anticancer drugs over a period of 7 days. Heterogeneous 3D spheroids were isolated from PGMatrix™ and fixed with 10% formalin. Spheroids were embedded in histogel for histology process. 5 μm thickness sections were cut and immunohistochemistry assay was performed using DAPI, staining nuclei (blue), E-cadherin antibodies (green), β-catenin antibodies (yellow), and vimentin antibodies, staining endothelial cells (pink). FIG. 6 contains an image captured using an Aperio ImageScope of samples, treated with 0.5 μM Erlotinib and/or 0.5 μM Cediranib for 6 days.

### Example 2

Peptide cell linkers of small synthetic peptide sequences of 23 residues or less and comprising at least two different cell adhesion peptide sequences were synthesized. The strategy is to promote interaction and association of two or more different cell types for growth and expansion into heterogeneous spheroids. Thus, the first task is to synthesize peptides with these properties. The following chemically-synthesized peptides were made commercially by New England Peptides (NEP):

(SEQ ID NO: 5)
FLIYIGSRIGPGGDGPGGRGD
(SEQ ID NO: 9)
YIGSRIGPGPGGRGD
(SEQ ID NO: 10)
RGDIGPGGDGPGGRGD
(SEQ ID NO: 6)
FLIYIGSRIGPGGDGPGGKGD
(SEQ ID NO: 7)
FLIYIGSRIGPGGDGPGGLDV
(SEQ ID NO: 11)
LDVGPGGDGPGGRGD
(SEQ ID NO: 12)
FLIYIGSRIGPGGDGPGGYIGSR
(SEQ ID NO: 2)
IGPGGDGPGG

Fifty milligrams of each peptide were synthesized with ≥95% purity with standard analytical techniques in the field (HPLC/mass spectrometry), and a MW range of from 200-1500.

All 8 peptide were dissolvable in RPMI media. Peptides were also tested with 1 mM PGMatrix™ for any alteration in gelation. The results show that there is no observable change in the gelation of 1 mM PGMatrix™ with or without 1% peptide derivative. Thus, all 8 peptide derivatives were used in further experiments for culturing with human tumor cell lines.

### Example 3

The peptide cell linkers were successfully synthesized and dissolved in the conditioning media. Six different human tumor cell lines—MDA-MB-231, MDA-MB-468, DU145, COLO205, PANC-1, CAKI-1, and HT29—were grown in the presence and absence of the following peptide cell linkers:

| Cell | |
|---|---|
| linker | Sequence |
| 1 | FLIYIGSRIGPGGDGPGGRGD (SEQ ID NO: 5) |
| 2 | YIGSRIGPGPGGRGD (SEQ ID NO: 9) |
| 3 | RGDIGPGGDGPGGRGD (SEQ ID NO: 10) |
| 4 | FLIYIGSRIGPGGDGPGGKGD (SEQ ID NO: 6) |
| 5 | FLIYIGSRIGPGGDGPGGLDV (SEQ ID NO: 7) |
| 6 | LDVGPGGDGPGGRGD (SEQ ID NO: 11) |
| 7 | FLIYIGSRIGPGGDGPGGYIGSR (SEQ ID NO: 12) |
| 8 | IGPGGDGPGG (SEQ ID NO: 2) |

Cells were cultured with peptide cell linkers in PGMatrix™ at a 1:1 ratio in RPMI conditioning media. Each cell line in PGMatrix™ along with peptide cell linker was also co-cultured with human umbilical vein endothelial cells (HUVECs) at a 1:3 ratio of HUVEC:human tumor cells, respectively. Controls were cells grown in 1 mM PGMatrix™ RPMI media for 7 days with no cell linkers.

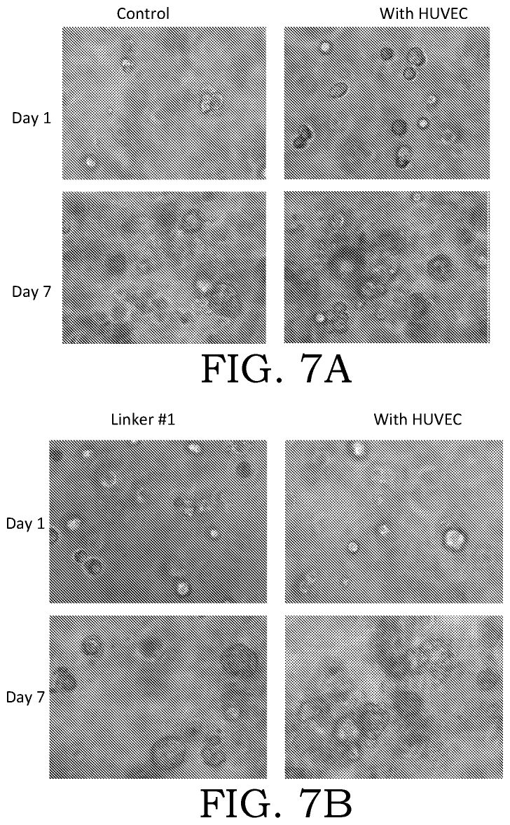

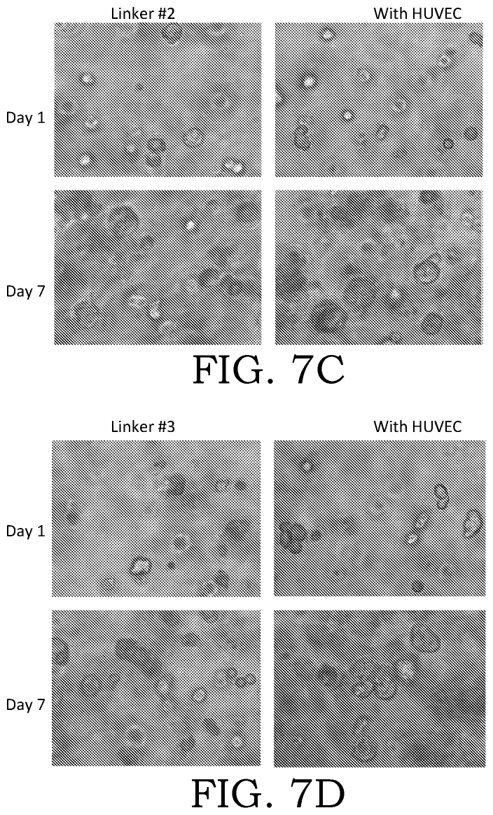

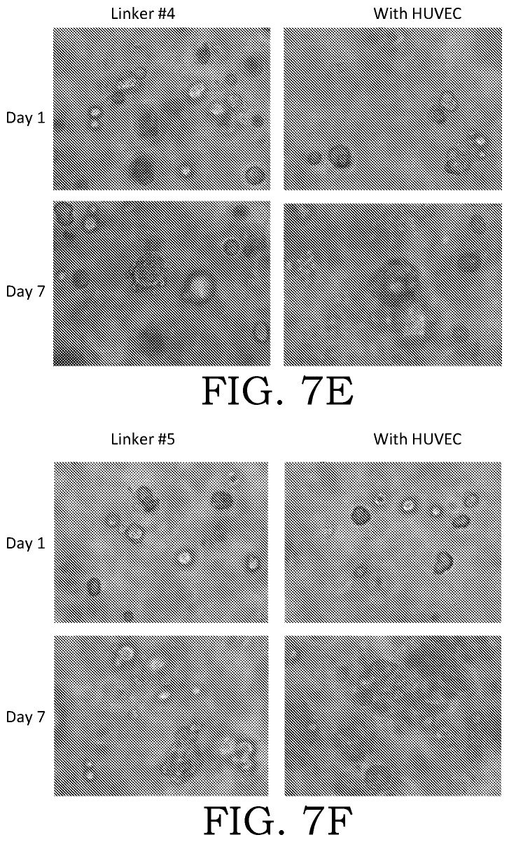

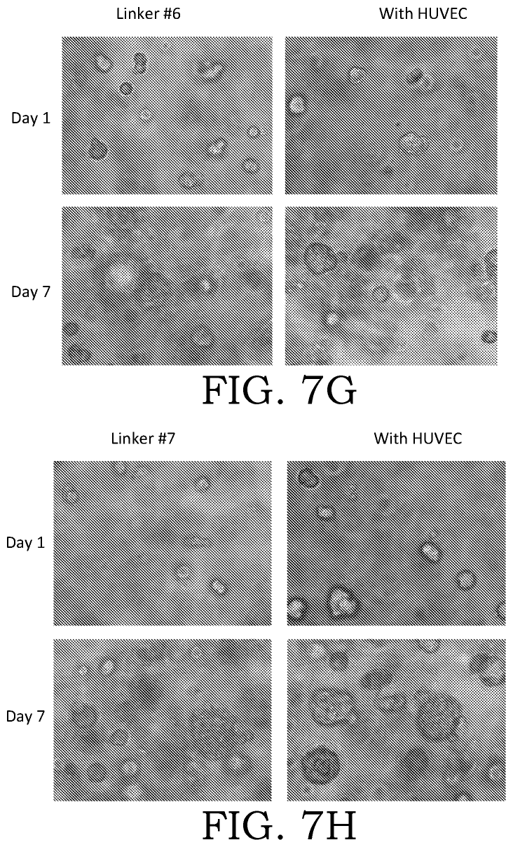

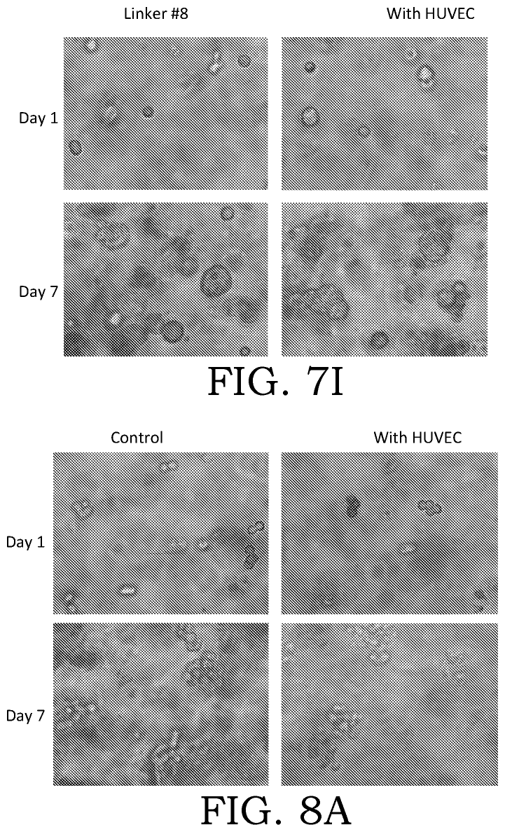

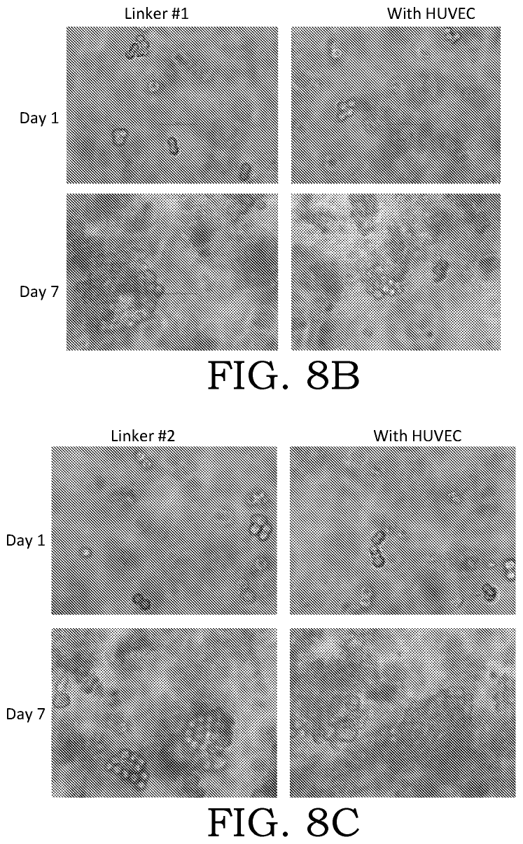

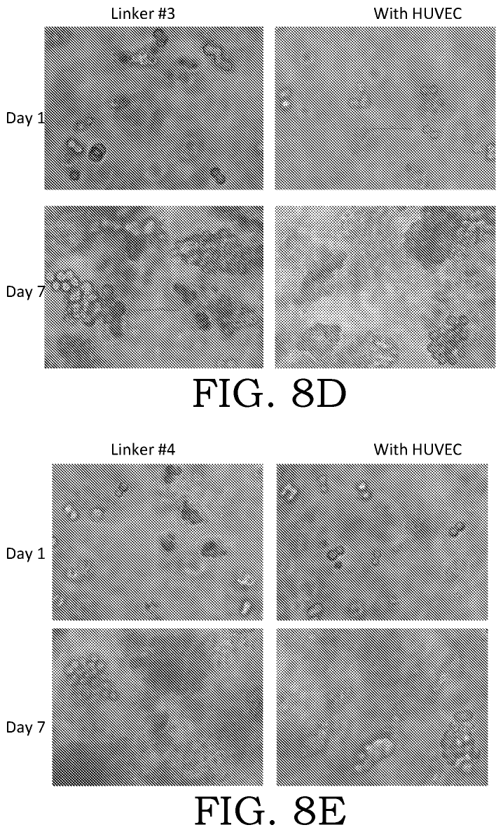

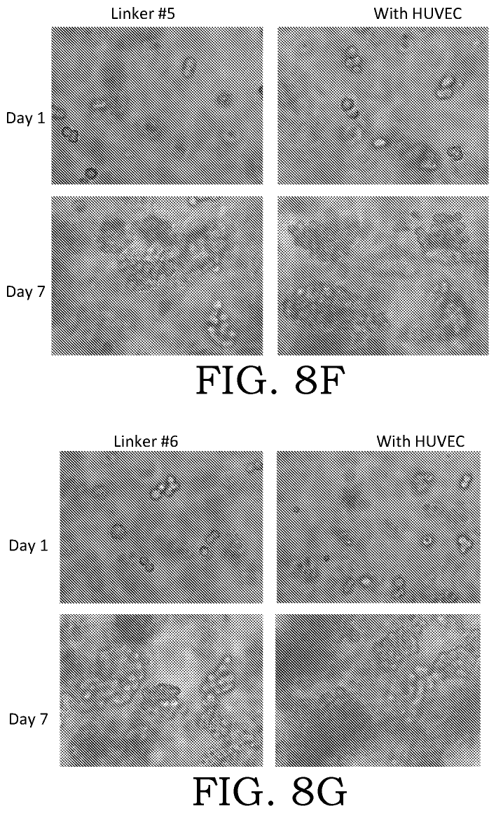

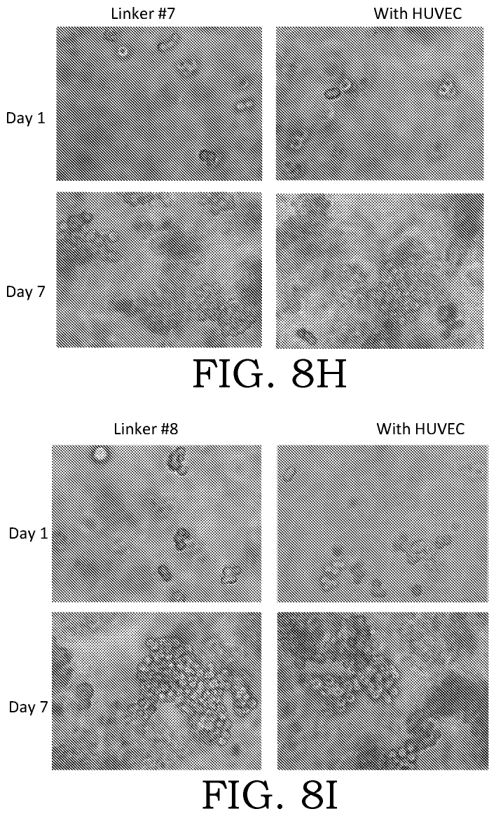

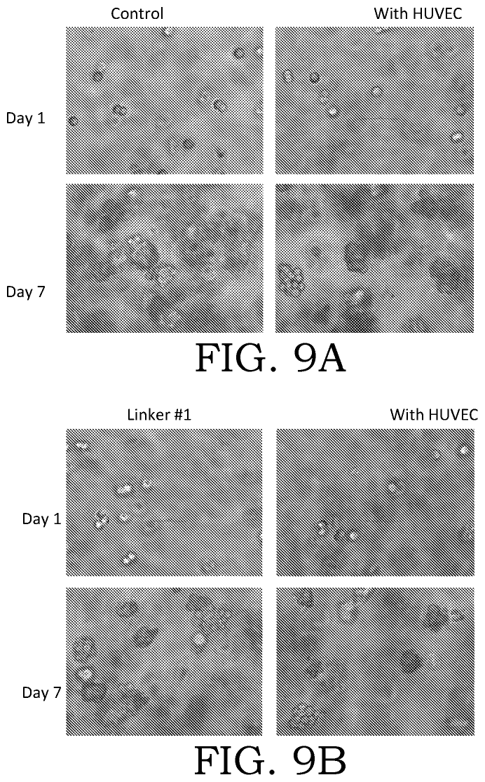

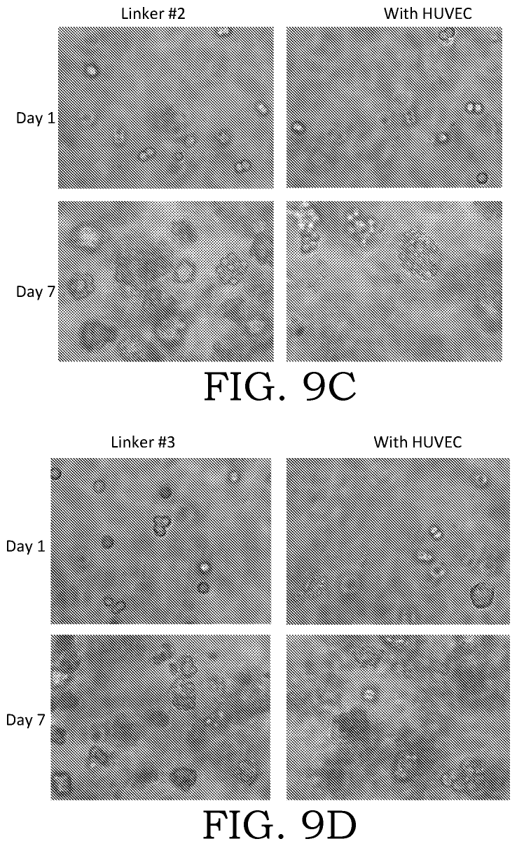

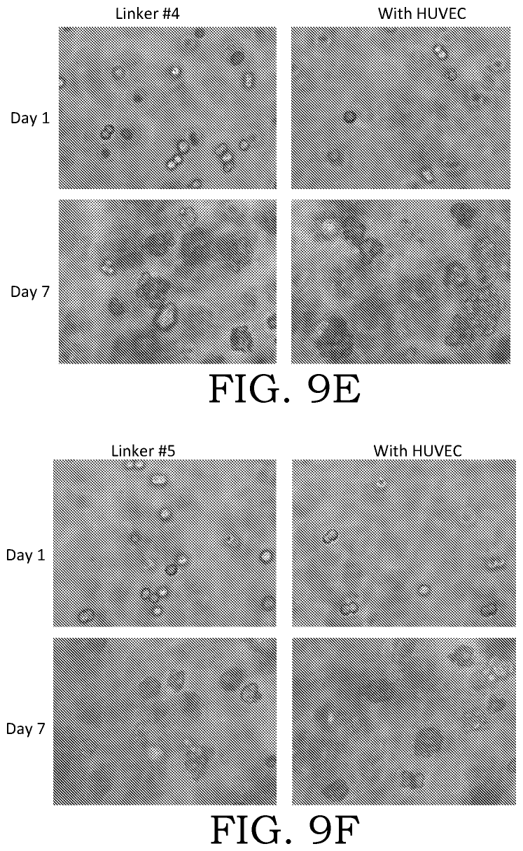

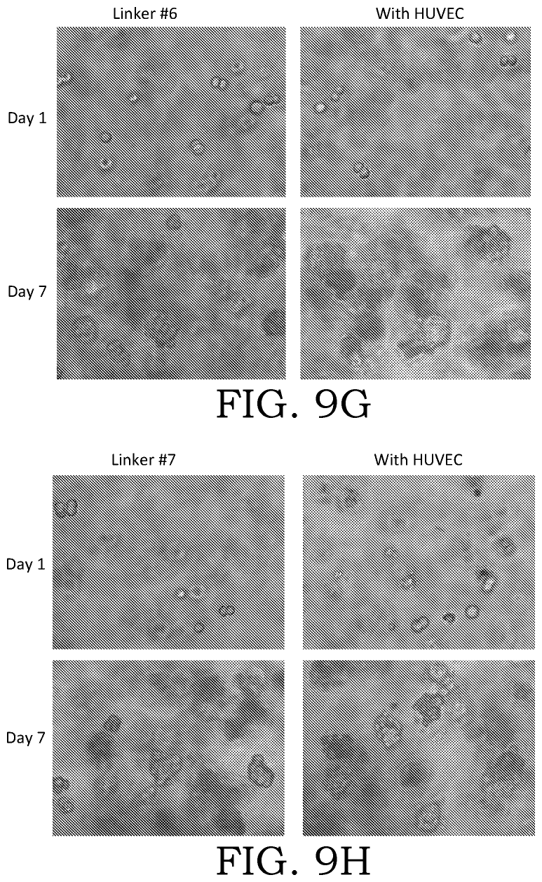

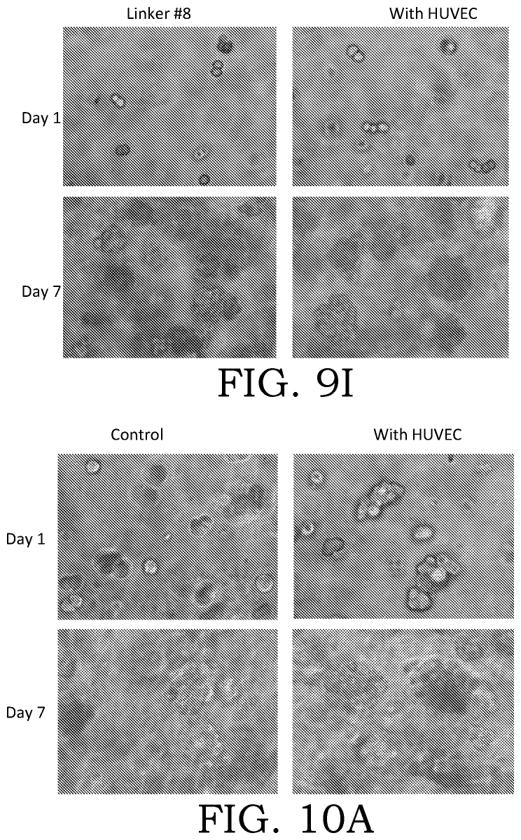

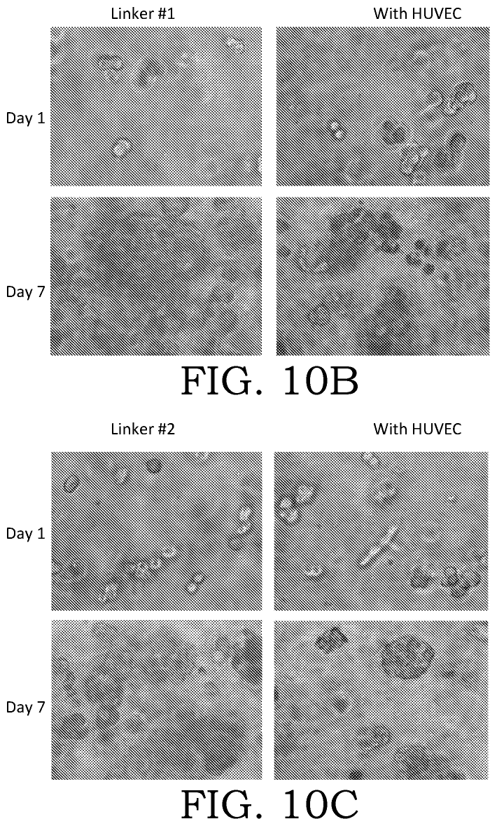

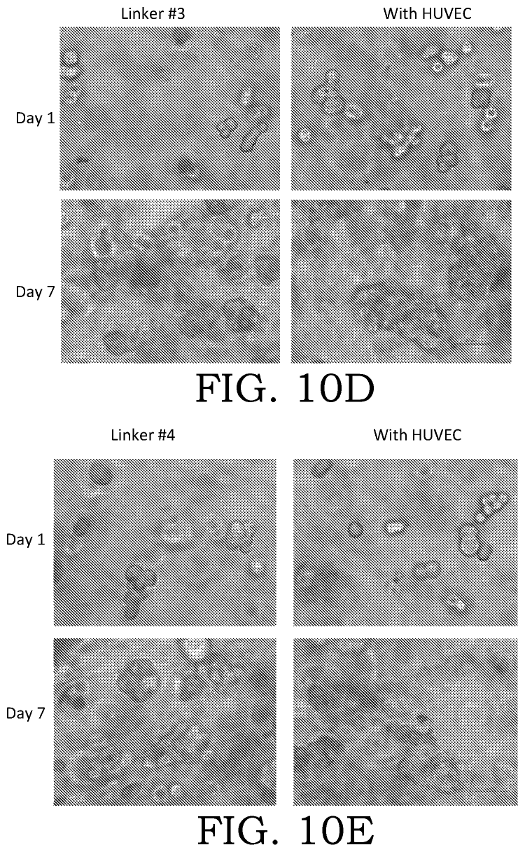

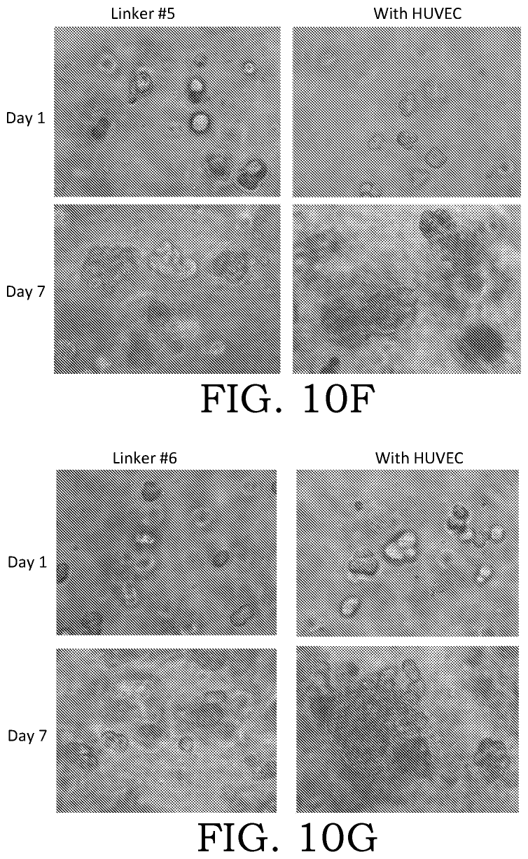

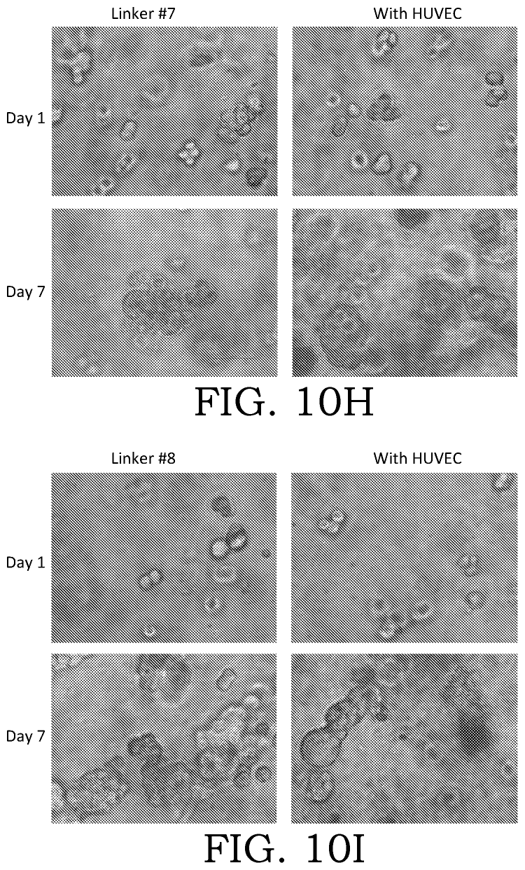

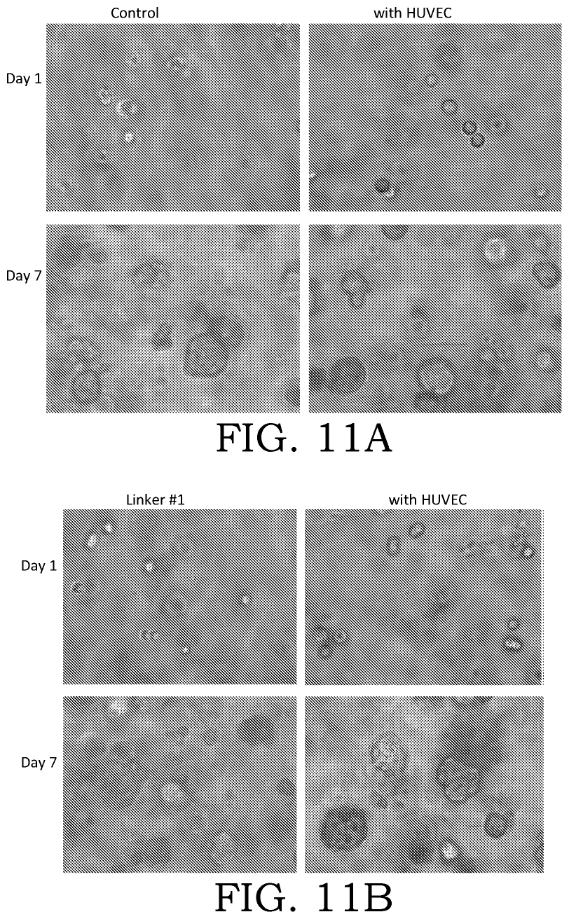

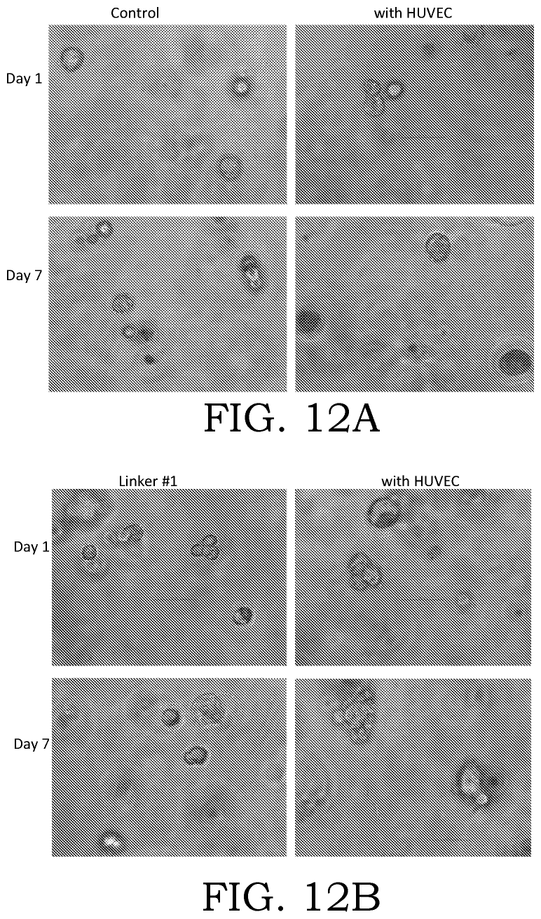

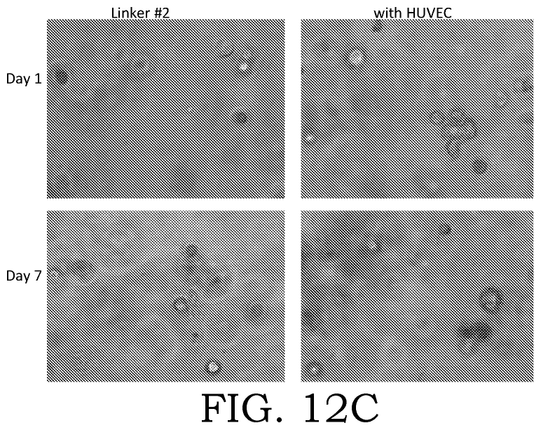

The resulting cultures were then imaged. Phase contrast microscopy was used and photomicrographs were obtained at Day 1 and Day 7 with or without HUVECs in the presence and absence of peptide cell linkers. The results are in FIGS. 7-12.

The figures demonstrate the support of 3D growth of three human tumor cell lines in PGMatrix™ with peptide cell linkers: DU145, MDA-MB-468, COLO205, PANC-1, CAKI-1, and HT29 cells. The results indicate that DU145 cells can form >50 μm spheroids in 1 mM PGMatrix™. Further, peptide cell linkers 4-7 increase the growth of these spheroids compared to controls and linker 8. For DU145, linkers 1-3 have the same rate of spheroid formation as controls with or without HUVEC. All peptide cell linkers enhanced the growth of MDA-MB-468 cells alone or with HUVECs compared to controls. Overall, linkers 1-7 promote growth depending on cell types and composition.

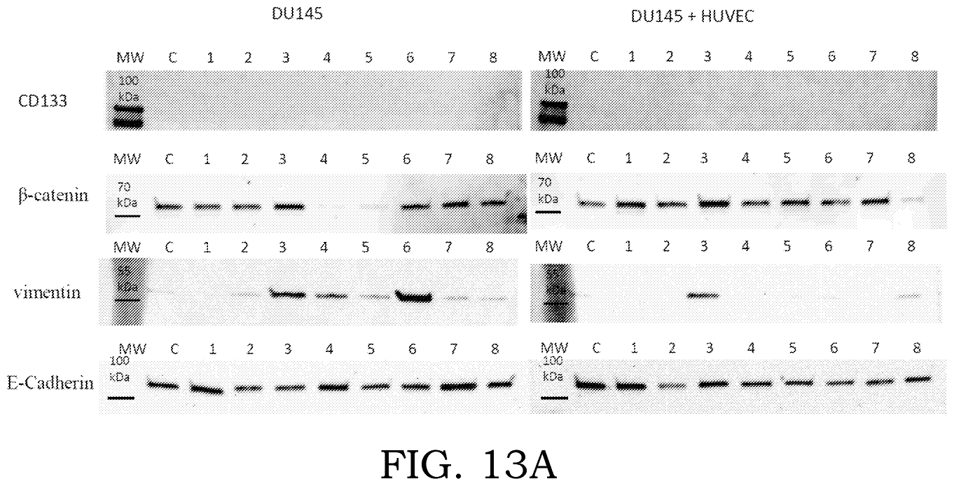

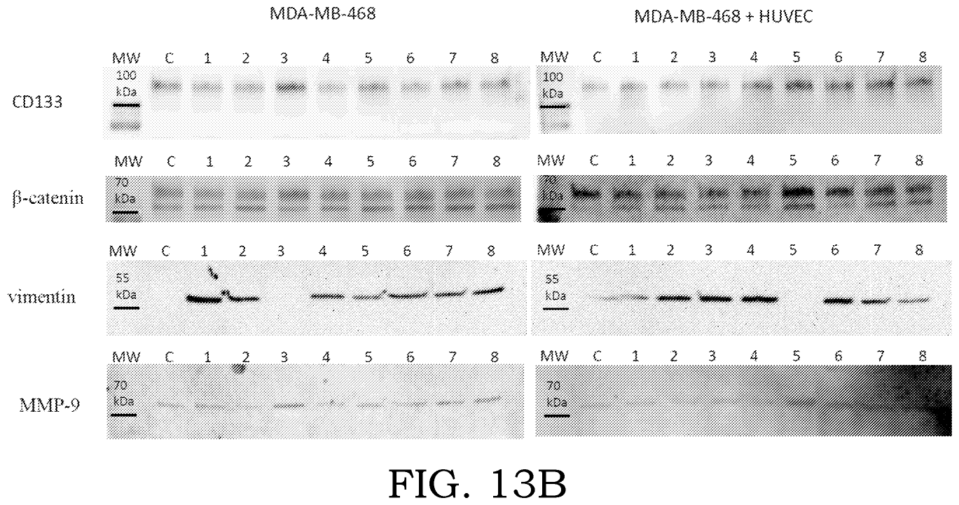

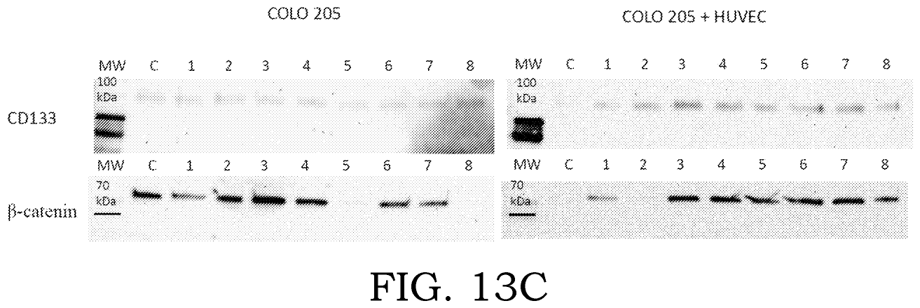

Western blot analysis was also performed on these spheroids for molecular signatures, i.e., vimentin, beta-catenin, and e-cadherin. Spheroids of human cancer cells were extracted from the PGMatrix-RPMI-linkers culture for biomarker analysis. Western blot analysis was performed against selected antibodies as indicated in the Figures (FIG. 13). Overall conclusion from these experiments is that the linkers exhibit a biologic function in promoting growth with cell specificity and molecular signature.

1. A three-dimensional heterogenous cell spheroid comprising a plurality of at least two different types of cells and one or more peptide cell linkers.

2. The spheroid of claim 1, wherein said cells proliferate in said spheroid.

3. The spheroid of claim 1, wherein each of said peptide cell linkers is a linear peptide having respective terminal ends (an N-terminal end and a C-terminal end), and comprising an adhesion sequence at or near each end.

4. The spheroid of claim 3, wherein each of said peptide cell linkers further comprises a spacer sequence between each adhesion sequence.

5. The spheroid of claim 4, wherein each spacer sequence comprises IGPGGDGPGG (SEQ ID NO:2), IGPGPGG (SEQ ID NO:3), or GPGGDGPGG (SEQ ID NO:4).

6. The spheroid of claim 3, wherein each adhesion sequence comprises YIGSR (SEQ ID NO:1), RGD, KGD, or LDV.

7. The spheroid of claim 3, wherein the adhesion sequence at one end of said peptide is different from the adhesion sequence at the other end of said peptide.

8. The spheroid of claim 3, wherein the adhesion sequence at one end of said peptide recognizes a first cell type and the adhesion sequence at the other end of said peptide recognizes a second cell type.

9. The spheroid of claim 3, wherein said adhesion sequences are each within 5 amino acid resides from their respective terminal end of said peptide.

10. The spheroid of claim 1, wherein said peptide cell linkers each consist of 25 or less amino acid residues.

11. The spheroid of claim 1, wherein said different cell types are selected from the group consisting of epithelial cells, stromal cells, endothelial cells, mesenchymal cells, stem cells, tumor cells, fibroblasts, adipocytes, dendritic cells, macrophages, and combinations thereof.

12. The spheroid of claim 1, said spheroid comprising at least one cancer cell type and at least one non-cancer cell type.

13. The spheroid of claim 1, wherein said different cell types are uniformly distributed throughout the body of said spheroid.

14. The spheroid of claim 1, said spheroid having a diameter of from about 50 to about 500 microns.

15. A composition comprising a hydrogel comprising a plurality of spheroids according to claim 1 distributed therein.

16. A method of forming a spheroid according to claim 1, said method comprising:
preparing a cell suspension comprising said plurality of at least two different types of cells in a cell culture medium;
mixing said one or more peptide cell linkers with said cell suspension to yield a cell mixture;
adding said cell mixture to a cell culture environment; and
culturing said cell mixture in said cell culture environment under favorable conditions for cell proliferation and spheroid formation in said cell culture environment, wherein each of said spheroids comprises said plurality of at least two different types of cells and one or more peptide cell linkers.

17. The method of claim 16, wherein said cell culture environment is a hydrogel matrix, said method comprising culturing said cell mixture in said hydrogel matrix under favorable conditions for cell proliferation and spheroid formation in said hydrogel.

18. The method of claim 17, wherein said adding said cell mixture to a hydrogel matrix comprises:
combining said cell mixture with a peptide suspension comprising a plurality of hydrogel-forming peptides; and
allowing said peptide suspension to form a hydrogel having said cells dispersed therein.

19. The method of claim 17, wherein the hydrogel-forming peptides are amphiphilic and self-assembling linear peptides.

20. The method of claim 17, wherein said peptide cell linkers are soluble in said hydrogel matrix.

21. The method of claim 17, wherein said hydrogel is shear thinning.

22. The method of claim 17, further comprising collecting said spheroids from said hydrogel matrix.

23. The method of claim 22, wherein said collecting comprises applying a kinetic force to said hydrogel matrix to reverse said hydrogel and release said spheroids.

24. An in vitro test method comprising:
providing a spheroid comprising a plurality of at least two different types of cells and one or more peptide cell linkers according to claim 1;
contacting said spheroid with a compound of interest for a period of time; and
detecting the effect of said compound of interest on said spheroid or the cells therein.

25. The method of claim 24, wherein said spheroid comprises at least one type of cancer cell, wherein said compound of interest is a chemotherapeutic, and wherein at least one effect of said compound of interest on said spheroid is cell death.

26. The method of claim 25, wherein said cancer cell is collected from a patient and used to form said spheroid prior to said test.

27.-29. (canceled)

30. The method of claim 24, said method comprising:
providing a plurality of said spheroids;
contacting said spheroids with a plurality of compounds of interest for a period of time, each of said spheroids being contacted with a respective compound of interest and/or defined combination of compounds of interest from a compound library; and
detecting the effect of said compounds of interest on said spheroids or the cells therein.


SEQUENCE LISTING (14 sequences)

SEQ ID NO: 1 - protein, 5 aa, Artificial Sequence
  Synthetic adhesion sequence
YIGSR

SEQ ID NO: 2 - protein, 10 aa, Artificial Sequence
  Synthetic spacer sequence
IGPGGDGPGG

SEQ ID NO: 3 - protein, 7 aa, Artificial Sequence
  Synthetic spacer sequence
IGPGPGG

SEQ ID NO: 4 - protein, 9 aa, Artificial Sequence
  Synthetic spacer sequence
GPGGDGPGG

SEQ ID NO: 5 - protein, 21 aa, Artificial Sequence
  Synthetic peptide linker
FLIYIGSRIGPGGDGPGGRGD

SEQ ID NO: 6 - protein, 21 aa, Artificial Sequence
  Synthetic peptide linker
FLIYIGSRIGPGGDGPGGKGD

SEQ ID NO: 7 - protein, 21 aa, Artificial Sequence
  Synthetic peptide linker
FLIYIGSRIGPGGDGPGGLDV

SEQ ID NO: 8 - protein, 16 aa, Artificial Sequence
  Synthetic peptide linker
LDVIGPGGDGPGGRGD

SEQ ID NO: 9 - protein, 15 aa, Artificial Sequence
  Synthetic peptide linker
YIGSRIGPGPGGRGD

SEQ ID NO: 10 - protein, 16 aa, Artificial Sequence
  Synthetic peptide linker
RGDIGPGGDGPGGRGD

SEQ ID NO: 11 - protein, 15 aa, Artificial Sequence
  Synthetic peptide linker
LDVGPGGDGPGGRGD

SEQ ID NO: 12 - protein, 23 aa, Artificial Sequence
  Synthetic peptide linker
FLIYIGSRIGPGGDGPGGYIGSR

SEQ ID NO: 13 - protein, 16 aa, Artificial Sequence
  Synthetic peptide linker
RGDIGPGGDGPGGLDV

SEQ ID NO: 14 - protein, 19 aa, Artificial Sequence
  Synthetic hydrogel peptide
FLIVIGSIIGPGGDGPGGD